(19) United States
(12) Patent Application Publication
(10) Pub. No.: US 2015/0106964 A1
(43) Pub. Date: Apr. 16, 2015

(54) METHODS FOR PRODUCING ORGANISMS CAPABLE OF INGESTING AND DIGESTING OMEGA-3 RICH SOURCES IN GREATER VOLUME AND OMEGA-3 ENRICHED HYBRID ORGANISMS

(71) Applicant: Sadia Ross Barrameda, Discovery Bay, CA (US)

(72) Inventor: Sadia Ross Barrameda, Discovery Bay, CA (US)

(21) Appl. No.: 14/579,943

(22) Filed: Dec. 22, 2014

Related U.S. Application Data
(62) Division of application No. 13/694,762, filed on Jan. 3, 2013.

(51) Int. Cl. C12N 15/90

(52) U.S. Cl. CPC C12N 15/90
USPC 800/25; 800/21

(57) ABSTRACT

The methods of producing an organism capable of ingesting and digesting omega-3 rich sources; for producing an organism that desires the consumption of omega-3 rich sources; and for producing an omega-3 enriched hybrid organism are each described. Each method isolates a donor DNA/RNA strand of a donor organism; extracts the donor DNA/RNA strand from the donor organism; and fuses the donor DNA/RNA strand into a receiving DNA/RNA strand of a receiving organism. In the first method, the donor organism is capable of ingesting and digesting omega-3 rich sources, and the receiving organism is incapable of ingesting or digesting omega-3 rich sources. In the second method, the donor organism desires the consumption of omega-3 rich sources, and the receiving organism does not desire the consumption of omega-3 rich sources. In the third method, the donor organism produces omega-3 fatty acids, and the receiving organism is unable to produce omega-3 fatty acids.

### CROSS-REFERENCE TO RELATED APPLICATION

The present application is a divisional of, and claims priority to, U.S. patent application Ser. No. 13/694,762 filed on Jan. 3, 2013, entitled “Methods of Producing New Types of Hybrid Silk and Fibers Using Insects, Animals, and Plants” the entire disclosure of which is incorporated by reference herein.

### FIELD OF THE INVENTION

The field of this invention relates generally to methods for producing animals that are capable of either consuming or internally producing Omega-3 fatty acids, thereby producing higher quality meat for human consumption. More specifically, this invention relates to the isolation and extraction of DNA and RNA from organisms that either produce Omega-3 fatty acids, or organisms that can and/or desire to consume (ingest and digest) hemp, and then fusing that DNA and RNA into other animals or organisms that contain small amounts of Omega-3 fatty acids.

### BACKGROUND OF THE INVENTION

The health benefits of long chain omega-3 (n-3) fatty acids are well recognized. Meat products normally contain small amounts of n-3 fatty acids and large amounts of n-6 fatty acids. Diets with a high ratio of n-6/n-3 fatty acids may contribute to the prevalence of many diseases, such as coronary artery disease, cancer, diabetes, arthritis and depression. The high n-6/n-3 ratio in meat products is largely due to the extensive use of grains rich in n-6 fatty acids but deficient in n-3 fatty acids as animal feed. In addition, livestock cannot convert n-6 fatty acids into n-3 fatty acids because they lack an n-3 fatty acid desaturase gene, such as the fat-1 gene found in certain organisms, such as the roundworm, C. elegans.

While meat products are generally low in n-3 fatty acids, cold water oily fish such as salmon, herring, mackerel, anchovies, and sardines have a high content of n-3. These fish, however, do not synthesize the n-3, but rather obtain them from the algae (microalgae in particular) or plankton in their diets.

Organisms can certainly be capable of feeding on hemp, like birds such as pigeons and doves feed on hemp seeds, for example. Certain animals, insects and pests, however, are drawn to eating hemp. Some organisms even feed on hemp solely, such as the hemp borer (Grapholita delineana).

While work has been done on cloning animals with the fat-1 gene that allows certain animals to convert n-6 fatty acids into n-3 fatty acids, there is little prior art regarding isolating, extracting, and fusing genes of organisms via transfection that would make animals that do not normally consume hemp, to consume (ingest and digest) hemp in greater volume. Furthermore, there is little prior art on using transfection to isolate and extract the DNA and RNA of Omega-3 producing organisms, and then fusing those genes into other animals, allowing those animals to produce Omega-3.

Therefore, there is a need for producing organisms and/or animals that are inclined to consume and capable of ingesting and digesting Omega-3 rich foods, such as hemp. In addition, there is a need for producing organisms and/or animals capable of internally producing Omega-3.

### SUMMARY OF THE INVENTION

This method uses existing hemp plants as a basis as well as other types of caterpillars, insects, including spiders, animals and bioreactors to produce new types of animals and organisms.

The end result is new combinations of DNA, in both plants and animals, using grafting, crossbreeding, splicing, genetic engineering, bioengineering and using all known and future methods and techniques creating new, different and improved, stronger, more attractive, usable fabrics, materials, fibers, dietary supplements, meats, for the textile, fashion, materials, fiber, food and seed industries.

The process isolates and extracts the DNA from Omega-3 producing organisms and fusing that DNA into the genome of chickens, goats and other animals. This process further isolates and extracts the DNA from the genes of organisms prone to consuming hemp, causing those organisms to ingest and digest hemp plants and seeds so that they will digest (and retain more omega 3 and other beneficial elements) in greater volume. The hemp plants and seeds create richer omega 3 meat in their meat.

A method for producing an organism capable of ingesting and digesting omega-3 rich sources is described, comprising the steps of isolating a donor DNA/RNA strand of a donor organism capable of ingesting and digesting omega-3 rich sources; extracting the donor DNA/RNA strand from the donor organism; and fusing the donor DNA/RNA strand into a receiving DNA/RNA strand of a receiving organism incapable of ingesting or digesting omega-3 rich sources.

In an embodiment, hemp is added to a diet of the receiving organism until the receiving organism can digest between 1% and 99% of the diet.

In a further embodiment, the omega-3 rich sources are selected from the group consisting of industrial hemp, cannabis and marijuana.

In a further embodiment, the donor DNA/RNA strand fuses into the receiving DNA/RNA strand by a means selected from the group consisting of chemical-based transfection, electroporation, sono-poration via transfection, optical transfection, gene electro transfer technique, impalefection, hydrodynamic delivery, magnetofection, using one or more viruses as a carrier, nucleofection, and transient transfection.

A method for producing an organism that desires the consumption of omega-3 rich sources is described, comprising the steps of isolating a donor DNA/RNA strand of a donor organism wherein the donor organism desires the consumption of omega-3 rich sources; extracting the donor DNA/RNA strand from the donor organism; and fusing the donor DNA/RNA strand into a receiving DNA/RNA strand of a receiving organism, wherein the receiving organism does not desire the consumption of omega-3 rich sources.

In an embodiment, hemp to a diet of the receiving organism until the receiving organism can digest between 1% and 99% of the diet.

In a further embodiment, the omega-3 rich sources are selected from the group consisting of industrial hemp, cannabis and marijuana.

In a further embodiment, the donor DNA/RNA strand fuses into the receiving DNA/RNA strand by a means selected from the group consisting of chemical-based transfection, electroporation, sono-poration via transfection, optical transfection, gene electro transfer technique, impalefection, hydrodynamic delivery, magnetofection, using one or more virus as a carrier, nucleofection, and transient transfection.

A method for producing an omega-3 enriched hybrid organism is described, comprising the steps of isolating a donor DNA/RNA strand of a donor organism; extracting the donor DNA/RNA strand from the donor organism; and fusing the donor DNA/RNA strand into a receiving DNA/RNA strand of a receiving organism, wherein the donor organism produces omega-3 fatty acids and the receiving organism is unable to produce omega-3 fatty acids.

In an embodiment, hemp is added to a diet of the receiving organism until the receiving organism can digest between 1% and 99% of the diet.

In a further embodiment, the donor DNA/RNA strand fuses into the receiving DNA/RNA strand by a means selected from the group consisting of chemical-based transfection, electroporation, sono-poration via transfection, optical transfection, gene electro transfer technique, impalefection, hydrodynamic delivery, magnetofection, using one or more virus as a carrier, nucleofection, and transient transfection.

### BRIEF DESCRIPTION OF THE FIGURES

For a more complete understanding of the present invention, the objects and advantages thereof, reference is now made to the ensuing descriptions taken in connection with the accompanying drawings briefly described as follows.

FIG. 1 is a flow chart of a method for producing an organism capable of ingesting and digesting omega-3 rich sources.

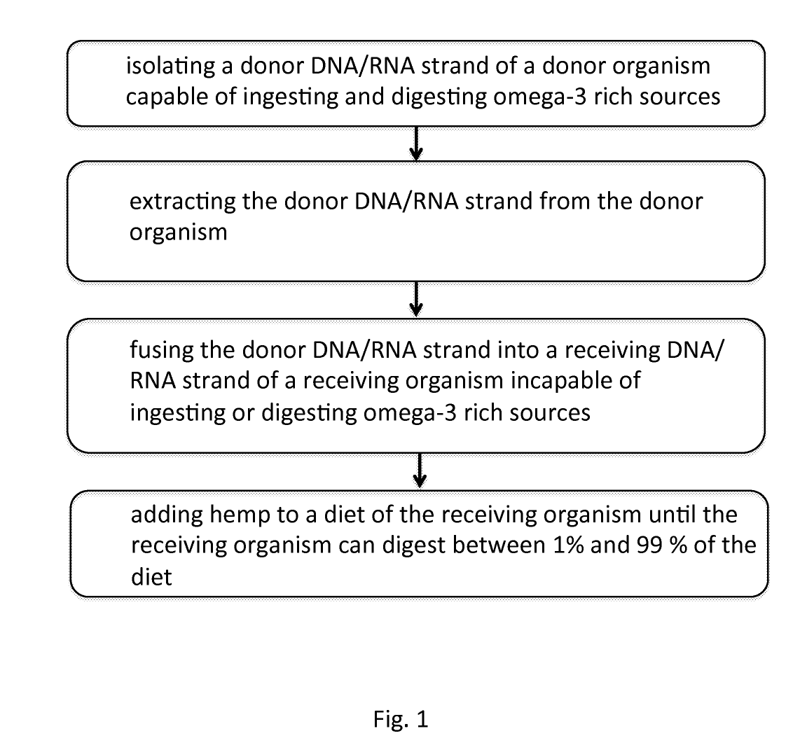

FIG. 2 is a flow chart of a method for producing an organism that desires the consumption of omega-3 rich sources.

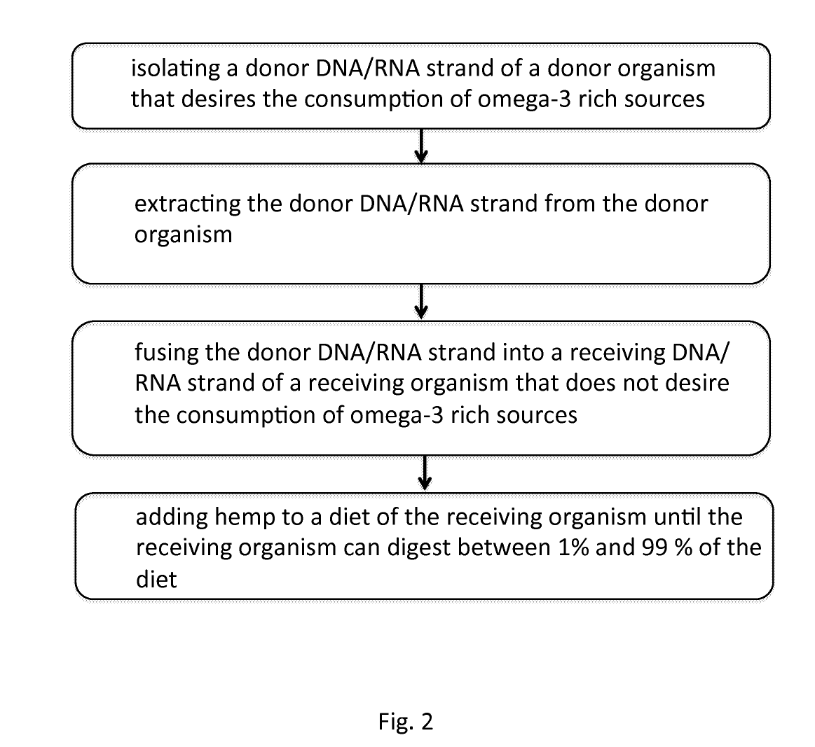

FIG. 3 is a flow chart of a method for producing an omega-3 enriched hybrid organism.

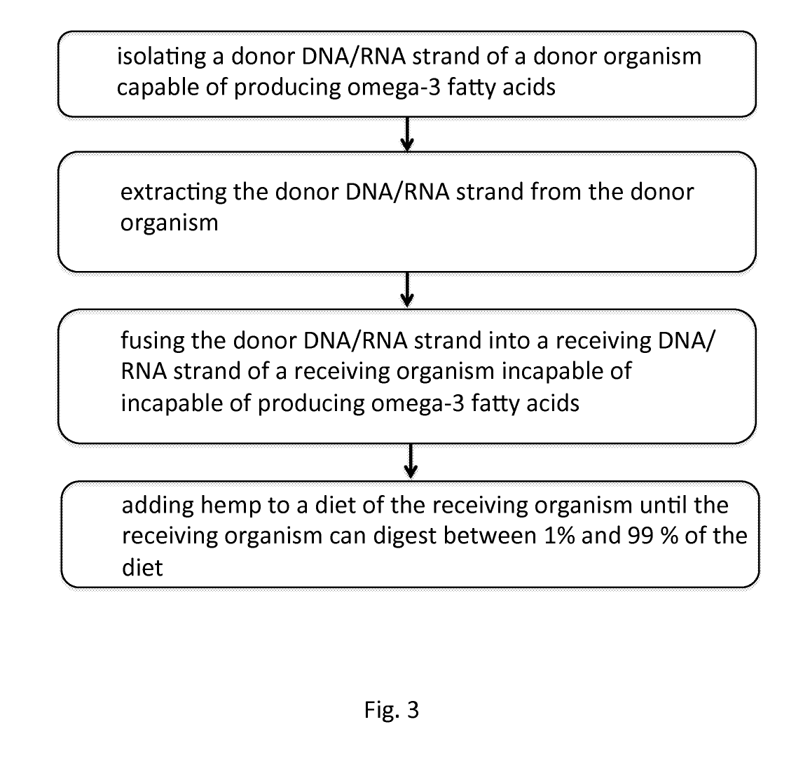

### DETAILED DESCRIPTION OF PREFERRED EMBODIMENTS

The method of the present invention can comprise any of the following steps alone, or in combination.

Adding hemp to the diet of organisms until they can digest between 1% to 99% of their diet in the various types of hemp, industrial hemp, including, excluding, but not limited to cannabis, marijuana.

Infusing the DNA AND/OR RNA of insects and rodents that normally eat hemp into that of other organisms, such as animals, to add to and/or increase hemp fibers in the meat of those organisms.

Growing various and new types of silk and cotton fibers in a lab utilizing the bio-reactor methods combining the DNA/RNA of the various types of hemp, cotton and silk worms, including but not limited to all plant types that produce fiber, i.e. cotton, hemp, that can be used to make clothing and/or fibers with that of the silk worm and other caterpillars, insects, spiders, rodents and animals.

Isolating and extract the DNA from Salmon and other omega 3 producing fish and animals and fusing same into the DNA, genome of chickens, goats and other animals. Isolating and extracting the DNA, genes of insects and animals that cause same to ingest and digest hemp and hemp seeds to create riche omega 3 meats in their bodies. The present invention refers to these new meats as “HEMP CHICKEN™, CHICKEN HEMP™, HEMPENCHICKEN™, “HEMP-FED CHICKEN™ and/or OMEGA 3 CHICKEN™ and applies these terms to all animals suitable for human consumption.

The following describes various methods and techniques for infusing the DNA of certain insects, animals and plants into other plants, animals and insects. Those methods may be used in all of these new combinations already described. However, the combinations may be achieved using other known and existing methods and techniques to combine same, and/or techniques and methods that will be discovered in the future. The primary uniqueness in these new ideas combine to create new plants and animals, and not necessarily the techniques and/or methods described below, which are primarily known methods and techniques for combining plants and animal forms of life.

The methods presented throughout this description may utilize the DNA of the 300 insect organisms or pests that cause those insects to ingest hemp and marijuana. Infusing the DNA and/or RNA of those organisms or pests into the DNA AND/OR RNA of other organisms or animals mentioned throughout this patent application causes them to ingest and/or digest hemp, especially the most serious hemp pests which are lepidopterist stem borers, predominately European corn borers (Ostrinia nubilalis) and hemp borers (Graptolitic delineana).

Utilize the below methods to infuse into other animal species the DNA AND/OR RNA that cause Beetle grubs that bore into stems and roots (e.g., Psylliodes attenuate, Ceutorhynchus rapae, Rhinocus pericarpius, Thyestes gebleri, and several Mordellistena spp.) to ingest and digest hemp.

Utilize the below methods to infuse into animal species the DNA and/or RNA that causes those insects to damage the field crops of leaves and flowering tops caused by caterpillars (e.g., hemp borers and budworms), beetles (e.g., Psylliodes attenuata), bugs, and leaf miners, including the predominant pests in marijuana crops such as the insects with piercing-sucking mouthparts, such as aphids (Pheromone Cannabis, Myzus Persicae, Aphis Fabae), whiteflies (Trialeurodes Vaporariorum, Bemisia spp.), Leafhoppers, and Mealy bugs.

Utilize the methods described below to infuse into organisms or animals the DNA AND/OR RNA that causes non-insect pests such as mites (Tetranychus Urticae, Aculops Cannabicola) to ingest and digest hemp. Utilize various methods of introducing foreign DNA AND/OR RNA of the hemp eating insects into a eukaryotic cell of other animals: sometimes relying on physical treatment (Electroporation, Nanoparticles, Magnetofection), other times on chemical materials or biological particles (viruses) that are used as carriers.

### Utilizing Chemical-Based Transfection

Chemical-based transfection will be divided into several kinds: cyclodextrin, polymers, liposomes or nanoparticles (with or without chemical or viral functionalization).

Use calcium phosphate, HEPES-buffered saline solution (HeBS) containing phosphate ions combined with a calcium chloride solution containing the DNA AND/OR RNA of the hemp eating insects to transfect the silk worms (and other animals). When the two are combined, a fine precipitate of the positively charged calcium and the negatively charged phosphate will form, binding the DNA AND/OR RNA to be transfected on its surface. The suspension of the precipitate is then added to the cells to be transfected (usually a cell culture grown in a monolayer). The cells of the silk worm (and other animals) take up some of the precipitate, and with it, the DNA AND/OR RNA.

In order to develop new silk (and all animal) producing species, one may use highly branched organic compounds, so-called dendrimers to bind the DNA AND/OR RNA and get it into the cell of any organism or animal.

Utilize the inclusion of the DNA AND/OR RNA to be transfected in liposome's, small, membrane-bounded bodies that are in some ways similar to the structure of a cell and can actually fuse with the cell membrane, releasing the DNA AND/OR RNA of the hemp eating insect into the cells of any prganism or animal. For eukaryotic cells, transfection is better achieved using cationic liposome's (or mixtures), because the cells are more sensitive.

Use cationic polymers such as DEAE-dextran or, polyethlyenimine. The negatively charged DNA AND/OR RNA of the hemp eating insects binds to the polycation of the silk worm (and all animal species) and the complex is taken up by the cell of all animal species) via endocythosis.

### Non-Chemical Methods

Utilize the electroporation method incorporating an instrument affecting the viability of many cell types creating micro-sized holes transiently in the plasma membrane of cells of the animal species or organism under an electric discharge. Similarly, utilize sono-poration via transfection applying sonic forces to the organisms or animal species cells.

Utilize the optical transfection method where a tiny (e.g., approximately 1 μm diameter) hole is transiently generated in the plasma membrane of the cells of the organism or animal using a highly focused laser. In this technique, one cell at a time is treated, making it particularly useful for single cell analysis.

Utilize the gene electro transfer technique enabling transfer of genetic material into the prokaryotic or eukaryotic cells of the organism. Basing this method on electroporation, a physical method where transient increases in the permeability of the organisms cell membrane are achieved when submitted to short and intense electric pulses.

Utilize the impalefection method of introducing hemp eating insect DNA AND/OR RNA bound to a surface of a nanofiber that is inserted into a cell of the organism or animal species. This approach can also be implemented with arrays of nanofibers that are introduced into large numbers of cells and intact tissue of the organism or animal species.

Utilize the hydrodynamic delivery of the DNA AND/OR RNA of hemp eating rodents, most often in their plasmids, including transposons, into organism or animal species antimicrobial peptides in the fat body (the insect equivalent of the vertebrate liver) using hydrodynamic injection that involves infusion of a relatively large volume in the blood in less than 10 seconds.

### Particle-Based Methods

Utilize the direct approach to transfection using the “gene gun” where the hemp eating insect's DNA AND/OR RNA is coupled to a nanoparticles of an inert solid (commonly gold) which is then “shot” directly into the organisms' or animal species' nucleus.

Utilize the magnetofection, or magnet assisted transfection, a transfection method, which will use magnetic force to deliver the hemp eating insect DNA AND/OR RNA into target cells of the organism or animal species. Nucleic acids are first associated with magnetic nanoparticles, then the application of magnetic force drives the nucleic acid particle complexes of the hemp eating insets towards and into the target cells of the organism or animal species, where the cargo is released.

Utilize impalefection, carrying this method out by impaling the organisms' or animal species' cells by elongated nanostructures and arrays of such nanostructures such as carbon nanofibers or silicon nanowires which have been functionalized with the plasmid DNA AND/OR RNA of the hemp eating insects.

### Viral Methods

Introduce the DNA AND/OR RNA of hemp eating insects into the organisms' or animal species' cells using viruses as a carrier. In such cases, the technique is called viral transcution, and the cells are said to be transduced. This can be done using insect cells.

Utilize methods of transfection, which include nucleofection, heat shock to infuse the DNA AND/OR RNA of hemp eating insects (and other hemp eating pests) into the cells of animal species.

### Stable and Transient Transfection

For many of the applications of transfection presented in this application, it is sufficient if the transfected genetic material is only transiently expressed. Since the DNA AND/OR RNA introduced in the transfection process is usually not integrated into the nuclear genome, the foreign DNA AND/OR RNA will be diluted through mitosis or degraded in the organism or animal species. When it is desired that the transfected gene actually remains in the genome of the cell and its daughter cells, a stable transfection may occur. To accomplish this, a marker gene is contransfected, which gives the cell some selectable advantage, such as resistance towards a certain toxin. Some of the transfected cells will, by chance, have integrated the foreign genetic material into their genome. If the toxin is then added to the cell culture, only those few cells with the marker gene integrated into their genomes will be able to proliferate, while other cells will die. After applying this selective stress (selection pressure) for some time, only the cells with a stable transfection remain and can be cultivated further into the organism or animal species.

Utilize geneticin as an agent for selecting stable transfection into the organism or animal species, also known as G418, which is a toxin that can be neutralized by the production of the neomycin resistance gene.

### RNA Transfection

Utilize transfected RNA into the organisms' or animal species' cells to transiently express its coded protein or to study RNA decay kinetics. The later application is referred as RNA transfection or RNA silencing, and has become a major application in research (to replace the “knock-down” experiments, to study the expression of proteins, i.e. of Endothelin-1) with potential applications in gene-therapy.

Utilizing the embedding of hemp protein [genes] sequenced within the organisms' or animal species' omega 3 [gene] sequences, causing those proteins to co-assemble into composite fibers in various increasing amounts of hemp protein (omega 3 for animals) starting at 1% up to 99%.

A small increase of hemp genes and omega 3 in the animals, the hybrid meat is richer than natural meat. To create these omega 3 rich animals, insert DNA from a hemp (or a type of hemp or cotton) eating organisms or pests into the embryonic cells of the various animals (or insert DNA from an omega 3 protein into the embryonic cells of the various animals). To make sure the genetic transformation is successful; add green fluorescent protein to the embryonic cells so that successfully engineered organisms and animals will glow under blue light.

Utilize all of the above methods to infuse the DNA into that of the organisms or animals. Utilize Cisgenic method to crossbreed by conventional means.

Utilize “knock out” method to knock out a dominant gene of an organism or animal. Another strategy is to attach the gene of hemp to a strong biological “promoter” of other elements and vice versa to see what happens when it is over expressed. Utilize the common technique to find out where the genes are expressed by attaching them to a “GUS reporter system” or a similar “reporter gene” that allows visualization of the location. Utilize RNA technology to match the inserted DNA of an endogenous gene already in the plant. When the inserted gene is expressed it can repress the “translation” of the endogenous protein. Utilize host delivered RNA systems so the plants will express RNA that will not interfere with animal species that have been engineered to have the appetite of the insects, nematodes and other parasites protein synthesis. This provides an additional novel way of enduring, the genetically engineered animal species to the genetically engineered hemp plants.

Utilize Mutagenesis to change the genetic information of the hemp and organisms in a stable manner, resulting in mutations. The results will occur both spontaneously and as a result of exposure to mutagens. This will also be achieved experimentally using laboratory procedures. Also, utilize the following techniques of spontaneous hydrolysis, cross linking, dimerization, and intercalation between bases, backbone damage, insertional mutagenesis, and intentional error in replication, mutagenesis as a laboratory technique, random mutagenesis and site-directed mutagenesis, combinatorial mutagenesis, insertional mutagenesis, and other types of mutagenesis.

Utilize molecular cloning to create recombinant DNA of all of the above combinations including polymerase chain reaction (PCR) used to direct the replication of any specific DNA sequence chosen by patent applicant. The present applicant herein may from time to time form recombinant DNA of the above mention using a cloning vector, a DNA molecule that will replicate within a living cell. Vectors will generally be derived from plasmids or viruses, and represent relatively small segments of DNA that contain necessary genetic signals for replication, as well as additional elements for convenience in inserting foreign DNA, identifying cells that contain recombinant DNA, and, where appropriate, expressing the foreign DNA. The choice when choosing to utilize vector for molecular cloning depends on the choice of host organism, the size of the DNA to be cloned, and whether and how the foreign DNA is to be expressed. The DNA segments will sometimes be combined by using a variety of methods, such as restriction enzyme/lipase cloning or Gibson assembly. The present applicant will normally utilize standard cloning protocols, the cloning of any DNA fragment essentially involving seven steps: (1) Choice of host organism and cloning vector, (2) Preparation of vector DNA, (3) Preparation of DNA to be cloned, (4) Creation of recombinant DNA, (5) Introduction of recombinant DNA into the host organism, (6) Selection of organisms containing recombinant DNA, (7) Screening for clones with desired DNA inserts and biological properties. Patent applicant will sometimes utilize molecular cloning to achieve commercialization of the ideas recorded throughout.

Create in a lab, using the bioreactor and/or the protein reactor techniques, attached to an artificial vine, or other apparatus, man-made silkworms that imitate the processes of spinning a cocoon so that as long as it is artificially fed with hemp and/or mulberry and/or any elements that will give man made silkworms such capacity to produce silk indefinitely, both of traditional consistency and of new improved strains of silk created from the unique combinations listed above throughout. The present invention includes using the same techniques of causing all other animals to consume hemp and transferring the DNA to those animals that will cause them to produce in their meats omega 3, as mentioned herein for chicken, to all consumable animals.

It is to be understood that the present invention is not limited in its application to the details of construction and the arrangement of the components set forth in the description. The invention is capable of other embodiments and of being practiced and carried out in various ways, also, it is to be fully understood that the phraseology and terminology employed herein are for the purpose of description and should not be regarded as limited. As such, those skilled in the art will appreciate that the conception, upon which this disclosure is based, may readily be utilized as a basis for the designing of other structures, methods and systems for carrying out the several purpose of the present invention. It is important, therefore, that the claims be regarded as including such equivalent constructions insofar as they do not depart from the spirit and scope of the present invention.

It is therefore an additional object of the present invention to provide a new and improved method for producing hybrid chickens and other animals fit for human consumption which have the advantage of the prior art devises and none of the disadvantages.

It is another object of the present invention to provide a new and improved method for producing hybrid animals, which may be easily and efficiently genetically modified, raised and marketed.

It is a further object of the present invention to provide a new and improved method for producing hybrid animals, which produce omega 3 in their bodies to enrich their dietary benefits for human consumption. An even further object of the present invention is to provide a new and improved method for producing hybrid animals which are susceptible of a low cost of production with regard to both materials and labor, and which accordingly is then susceptible of low prices of sale to the consuming public, thereby making such a method for producing hybrid animals and economically available to the buying public.

Still yet another object of the present invention is to provide a new and improved method for producing hybrid animals (both living for the farms and in a lab for bio-reactors) which provide in the apparatuses and methods of the prior art some of the advantages thereof, while simultaneously overcoming some of the disadvantages normally associated therewith.

Still another object of the present invention is to provide a new and improved method for producing hybrid animals operable from a novice's level.

Yet another object of the present invention is to provide a new and improved method for producing hybrid animals. These together with other objects of the present invention, along with the various features of novelty, which characterize the present invention, are pointed out with a particularity within the claims annexed in the utility patent.

I claim:

1. A method for producing an organism capable of ingesting and digesting omega-3 rich sources, comprising:
a. isolating a donor DNA/RNA strand of a donor organism capable of ingesting and digesting omega-3 rich sources;
b. extracting the donor DNA/RNA strand from the donor organism; and
c. fusing the donor DNA/RNA strand into a receiving DNA/RNA strand of a receiving organism incapable of ingesting or digesting omega-3 rich sources.

2. The method of claim 1, further comprising the step of adding hemp to a diet of the receiving organism until the receiving organism can digest between 1% and 99% of the diet.

3. The method of claim 1, wherein the omega-3 rich sources are selected from the group consisting of industrial hemp, cannabis and marijuana.

4. The method of claim 1, wherein the donor DNA/RNA strand fuses into the receiving DNA/RNA strand by a means selected from the group consisting of chemical-based transfection, electroporation, sono-poration via transfection, optical transfection, gene electro transfer technique, impalefection, hydrodynamic delivery, magnetofection, using one or more viruses as a carrier, nucleofection, and transient transfection.

5. A method for producing an organism that desires the consumption of omega-3 rich sources, comprising:
a. isolating a donor DNA/RNA strand of a donor organism wherein the donor organism desires the consumption of omega-3 rich sources;
b. extracting the donor DNA/RNA strand from the donor organism; and
c. fusing the donor DNA/RNA strand into a receiving DNA/RNA strand of a receiving organism,
wherein the receiving organism does not desire the consumption of omega-3 rich sources.

6. The method of claim 5, further comprising the step of adding hemp to a diet of the receiving organism until the receiving organism can digest between 1% and 99% of the diet.

7. The method of claim 5, wherein the omega-3 rich sources are selected from the group consisting of industrial hemp, cannabis and marijuana.

8. The method of claim 5, wherein the donor DNA/RNA strand fuses into the receiving DNA/RNA strand by a means selected from the group consisting of chemical-based transfection, electroporation, sono-poration via transfection, optical transfection, gene electro transfer technique, impalefection, hydrodynamic delivery, magnetofection, using one or more virus as a carrier, nucleofection, and transient transfection.

9. A method for producing an omega-3 enriched hybrid organism, comprising:
a. isolating a donor DNA/RNA strand of a donor organism;
b. extracting the donor DNA/RNA strand from the donor organism; and
c. fusing the donor DNA/RNA strand into a receiving DNA/RNA strand of a receiving organism,
wherein the donor organism produces omega-3 fatty acids and the receiving organism is unable to produce omega-3 fatty acids.

10. The method of claim 9, further comprising the step of adding hemp to a diet of the receiving organism until the receiving organism can digest between 1% and 99% of the diet.

11. The method of claim 9, wherein the donor DNA/RNA strand fuses into the receiving DNA/RNA strand by a means selected from the group consisting of chemical-based transfection, electroporation, sono-poration via transfection, optical transfection, gene electro transfer technique, impalefection, hydrodynamic delivery, magnetofection, using one or more virus as a carrier, nucleofection, and transient transfection.
